(19) United States
(12) Patent Application Publication
(10) Pub. No.: US 2002/0086903 A1
(43) Pub. Date: Jul. 4, 2002

(54) Synergistic biocidal oxidant

(76) Inventors: Charles J. Giambrone, New Hope, PA (US); George M. Diken, Hamilton, NJ (US)

Correspondence Address: Patent Administrator, FMC Corporation, 1735 Market Street, Philadelphia, PA, 19103, US

(21) Appl. No.: 09/742,799

(22) Filed: Dec. 21, 2000

Related U.S. Application Data
(60) Provisional application No. 60/244,274, filed on Oct. 30, 2000.

(51) Int. Cl. A61K 33/14; A01N 59/08; A61K 31/19

(52) U.S. Cl. 514/557; 424/661; 422/37

(57) ABSTRACT

A synergistic biocidal oxidant, useful as a sanitizer and disinfectant, is disclosed. The synergistic biocidal oxidant comprises a lower organic peracid, preferably peracetic acid, and chlorine dioxide.

### CROSS-REFERENCE TO RELATED APPLICATIONS

This application claims priority of U.S. Provisional Patent Application No. 60/244,274, filed Oct. 30, 2000.

### FIELD OF THE INVENTION

This invention relates to a synergistic biocidal oxidant. In particular, this invention relates to a synergistic mixture of a lower organic peracid and chlorine dioxide useful as a sanitizer and disinfectant.

### BACKGROUND OF THE INVENTION

Dilute, aqueous solutions of lower organic peracids, especially of peracetic acid, are effective against a wide spectrum of microorganisms, including algae, fungi, bacteria, and viruses. Because these peracids leave only the corresponding lower organic acids as residues, they are particularly suited for applications in which a non-environmentally-polluting disinfectant is required.

Chlorine dioxide has been used to treat drinking water because it produces lower levels of chlorinated hydrocarbons, such as trihalomethanes, than are produced by treatment with chlorine. Chlorine dioxide can also oxidize chlorophenols, produced by reaction of chlorine with phenolic compounds present in the water.

However, each of these reagents is relatively expensive to use as a large-scale disinfectant or sanitizer. Thus, a need exists for a composition that is an effective as a disinfectant or sanitizer at lower concentration so that less reagent is required.

### SUMMARY OF THE INVENTION

In one embodiment, the invention is a disinfectant composition. The composition comprises:

(a) water;

(b) about 10 ppm to about 500 ppm of a lower organic peracid; and

(c) about 0.1 ppm to about 20 ppm of chlorine dioxide.

In a preferred embodiment, the peracid is peracetic acid. The composition preferably comprises about 10 ppm to about 200 ppm, more preferably about 10 ppm to about 100 pmm, of the lower organic peracid, and about 0.4 ppm to about 2 ppm of chlorine dioxide. The disinfectant composition may also comprise hydrogen peroxide.

In another the embodiment, the invention is a method for disinfecting a surface by applying the composition to the surface.

### DETAILED DESCRIPTION OF THE INVENTION

In the specification and claims, unless the context indicates otherwise, all parts, percentages, and ratios are by weight and all temperatures are in °C. The terms “sanitizer,” “antimicrobial,” “disinfectant,” “biocidal” and similar terms are used interchangeably. Unless the context indicates differently, “peracid” refers to organic peracids and to mixtures of organic peracids.

Chlorine dioxide, ClO₂, is a well-known disinfectant for drinking water. Its properties and chemistry are described in The Chlorine Dioxide Handbook, D. J. Gates, American Water Works Association, Denver, 1998. Because it has an odd number of electrons, it is a free radical. It is a highly reactive species, which is extremely unstable at temperatures above −40° C. Aqueous solutions, however, are relatively stable when diluted at about 5 g/L or less and kept cold and away from strong light, such as direct sunlight. Chlorine dioxide reacts rapidly with any organic matter present in the water.

Chlorine dioxide can be generated by reaction of a chlorite, typically sodium chlorite, with an oxidizing agent, such as chlorine or hypochlorous acid, and/or with a strong acid, such as hydrochloric acid. For drinking water purposes, chlorine dioxide is formed almost universally by reacting sodium chlorite with an oxidizing agent and/or an acid in a mechanical generator. Depending on the generator design, the oxidizing agent may be: gaseous or aqueous chlorine alone; a strong acid, either alone or with chlorine; or an acid in combination with a hypochlorite salt solution. The principle by-products of generating and using chlorine dioxide are chlorite ion (ClO₂⁻), chlorate ion (ClO₃⁻), and chloride ion (Cl⁻). Electrochemical generation of chlorine dioxide from sodium chlorite has also been described. Numerous reactors and reaction schemes are known. These are summarized in The Chlorine Dioxide Handbook, D. J. Gates, American Water Works Association, Denver, 1998, Chapter 3.

The term “stabilized chlorine dioxide” is applied to a variety of formulations that claim to be aqueous solutions of chlorine dioxide stabilized in solution through a variety of complexes. Typically, they comprise a chlorite, typically sodium chlorite [NaClO₂], and activators that are designed to slowly release chloride dioxide from the mixture. Buffers may also be present to lower the pH of the solution. Use of these solutions avoids the need for the complex and costly equipment associated with chlorine dioxide generation. Chlorine dioxide can be generated from these solutions by reacting them with an acid, particularly a strong acid, if significant generation of chloride dioxide is required in a reasonable period of time.

“Peracid” and “organic peracid” refer to compounds of the structure R—CO—OOH, in which R is an organic radical. Although any organic peracid, or mixture of organic peracids, that has the requisite water solubility may be used in the composition, a lower organic peracid is preferred. Lower organic peracid refers to the peracid of an organic aliphatic monocarboxylic acid having 2 to 10 carbon atoms (i.e., R is an organic radical having from 1 to 9 carbon atoms), such as acetic acid (ethanoic acid), propionic acid (propanoic acid), butyric acid (butanoic acid), iso-butyric acid (2-methyl-propanoic acid), valeric acid (pentanoic acid), 2-methyl-butanoic acid, iso-valeric acid (3-methyl-butanoic acid), and 2,2-dimethyl-propanoic acid. Organic aliphatic peracids having 2 or 3 carbon atom are preferred. The most preferred organic peracid is peracetic acid (CH₃CO—OOH).

Mixtures of organic peracids may be used. For example, peracetic acid may be mixed with other lower organic acids and their corresponding peracids, such as with one or more peracids derived from aliphatic monocarboxylic acids having 3 to 10 carbon atoms (i.e. aliphatic monocarboxylic peracids having 3 to 10 carbon atoms), for example, perhexanoic acid, perheptanoic acid, per(2-ethyl) hexanoic acid, peroctanoic acid, pernonaoic acid, and/or perdecanoic acid. A preferred peracid for use with peracetic acid is peroctanoic acid (C₇H₁₅CO—OOH).

Organic peracids are formed from the corresponding organic acids and hydrogen peroxide by the following equilibrium reaction:

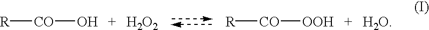

The equilibrium concentration of each reagent can be calculated from the equilibrium equation:

$$\frac{[ R - CO - OOH ] [ {H}_{2} O ]}{[ R - CO - OH ] [ {H}_{2} {O}_{2} ]} = {K}_{ap} \qquad ( II )$$

where:

[R—CO—OOH] is the concentration of peracid in mole/L;

[H₂O] is the concentration of water in mole/L;

[R—CO—OH] is the concentration of organic acid in mole/L;

[H₂O₂] is the concentration of hydrogen peroxide in mole/L; and

Kₐₚ is the apparent equilibrium constant for the peracid equilibrium reaction (Equation I).

The apparent equilibrium constant, Kₐₚ, is dependent on the peracid chosen and the temperature. Equilibrium constants for peracid formation are discussed in D. Swern, ed., Organic Peroxides, Vol. 1, Wiley-Interscience, New York, 1970. For peracetic acid at a temperature of 40° C., the apparent equilibrium constant is about 2.21.

In dilute solutions a relatively long period of time is required to attain equilibrium because of the low concentration of the reactants. Consequently, peracids are typically prepared in concentrated solution and then diluted to the required concentration prior to use. Thus, the disinfectant compositions typically additionally comprise about 5 ppm to about 5000 ppm, preferably about 10 ppm to about 1000 ppm, of hydrogen peroxide. In one embodiment, the disinfectant composition comprises about 50 pmm to about 500 pmm of hydrogen peroxide.

Equilibrium solutions that comprise about 5% peracetic acid typically comprise about 22% hydrogen peroxide. Equilibrium solutions that comprise about 15% peracetic acid typically comprise about 10% hydrogen peroxide. When these equilibrium solutions are diluted to solutions that comprise about 50 ppm of PAA, the solution produced by dilution of the 5% PAA solution comprises about 220 ppm of hydrogen peroxide, and the solution produced by dilution of 15% solution comprises about 33 ppm of hydrogen peroxide.

Organic peracid solutions also comprise the organic acid corresponding to the organic peracid and hydrogen peroxide. A catalyst, added to reduce the time required to reach equilibrium, is present some commercially available peracetic acid solutions. Typical catalysts are strong acids, such as, sulfuric acid, sulfonic acids, phosphoric, and phosphonic acids. When the peracid solution is diluted to produce the desired peracid level, the catalyst concentration is also reduced. The presence of low levels of sulfuric acid, for example concentrations in the range of about 1 ppm to about 50 ppm, does not adversely affect the properties of the composition.

Commercial organic peracid solutions typically contain a stabilizer. The stabilizer is a sequestering agent that chelates metals that catalyze the decomposition of hydrogen peroxide. These include, for example, pyridine carboxylates and organic phosphonic acids capable of sequestering bivalent metal cations, as well as the water-soluble salts of such acids. A common stabilizer is 1-hydroxyethylidene-1,1-diphosphonic acid, which is sold as DEQUEST® 2010 stabilizer. The low levels present in the composition after dilution do not significantly affect the properties of the composition. The composition may comprise other ingredients, such as colorants, which may be added so that the presence of the composition may be detected by visual inspection.

The disinfectant composition may be prepared by mixing an aqueous peracid solution, such as a solution comprising about 5% to about 35% by weight peracetic acid, and an aqueous solution of a chlorite salt, preferably sodium chlorite. “Stabilized chlorine dioxide” may also be used. Either an equilibrium or a non-equilibrium peracid solution, such as an equilibrium or non-equilibrium solution comprising about 4% to about 8% of peracetic acid, can be used. The resulting mixture is allowed to react for a predetermined period of time, typically about 0.25 min to about 5 min, preferably about 0.5 to about 3 min, and then diluted to a disinfectant composition of the desired peracid concentration. Dilution essentially stops the formation of chlorine dioxide. The concentration of hydrogen peroxide in the disinfectant composition will depend on the concentration in the starting PAA and the dilution necessary to produce the disinfectant composition with the desired PAA concentration.

The resulting disinfectant composition is used as described below. Because peracids are formed in equilibrium processes and the equilibrium reaction causes the concentration of peracid to slowly change after the concentrated peracid solution has been diluted, the disinfectant composition is preferably used soon after its preparation. Refrigeration may decrease the rate of the equilibrium processes and decrease the rate of concentration change in the disinfectant composition.

In one embodiment, the invention is a kit comprising two parts. The first part comprises a peracid solution, typically an aqueous solution of peracetic acid that is at or near equilibrium, typically comprising about 5% to about 35% by weight of peracid (on a 100% active basis). Mixtures of peracids, for example a mixture of peracetic acid and peroctanoic acid, may be used. The second part comprises sodium chlorite or “stabilized chlorine dioxide.” In use, the first part and second part are mixed together, allowed to react, and diluted as described above to produce the disinfectant composition.

Industrial Applicability

The disinfectant compositions are effective against a wide spectrum of microorganisms, including algae, fungi, bacteria, and viruses. They can be used, for example, to disinfect animal carcasses, meat products (such as are described in Gutzmann, U.S. Pat. Nos. 6,010,729 and 6,113,963), fruits and vegetables, medical instrument, dental instruments, food contact surfaces such as are found in food processing machinery and equipment, food, and hard surfaces, such as floors, counters, furniture, etc. such as are found in, for example, the food and health care industry, i.e., kitchens, restaurants, hospitals, clinics, nursing homes, doctors' and dentists' offices, medical laboratories, etc.

The disinfectant composition can also be used to disinfect a wide variety of fruits and vegetables, for example produce products such as head lettuce, leaf lettuce, radishes, celery, spinach, cabbage, carrots, beets, parsley, rhubarb, tomatoes, turnips, cauliflower, broccoli, Brussels sprouts, and dandelion greens; fruits such as apples, peaches, plums, grapes, and pears; and berries such as strawberries, raspberries, gooseberries, loganberries, boysenberries, blackberries, and blueberries. The disinfectant compositions can be applied by any method that insures good contact between the object to be disinfected and the disinfectant composition, for example, by coating, dipping, spraying, fogging, etc.

The composition can be used to disinfect a wide variety of animal carcasses such as: muscle meats such as beef, pork, veal, buffalo, lamb, venison, and mutton; seafood, such as scallops, shrimp, crab, octopus, mussels, squid, and lobster; and poultry such as chicken, turkey, ostrich, game hen, duck, goose, squab, and pheasant. “Animal carcass” refers to a portion of a carcass, for example an individual cut of meat, seafood, or poultry, as well as the entire carcass. Various techniques for applying the disinfectant composition to animal carcasses may be used. These techniques are generally disclosed in Gutzmann, U.S. Pat. No. 6,010,729, especially column 13, line 39, to column 16, line 20. These include, for example, spraying by a manual wand, spraying using multiple spray heads preferably in a spray booth, electrostatic spraying, fogging, and dipping or immersion preferably into an agitated solution. The composition may also be applied as a foam. These same techniques can be used to apply the disinfectant composition to other objects, for example fruits and vegetables.

The compositions are also useful as sanitizers in all types of industrial, food, dental, and medical transport and process water systems, including environmental remediation of biofouled water transport systems, such as cooling towers; pulp and paper process waters (“whitewater”); carrier streams for fruits, vegetables and other food products; and as a “clean-in-place” sanitizer and biofilm remover in industrial systems.

The disinfectant compositions may be used as sanitizers in aqueous food processing streams. After picking, fruits and vegetables are introduced into a flume system in which water acts as a transport medium and a cleaning medium. Transport water is used to support and transport the fruits or vegetables from an unloading location to a final storage or packing or processing location. Process water is used in some of the processing stages to further clean, cool, heat, cook, or otherwise modify the food in some fashion prior to packaging. In either situation, the water becomes contaminated with organic matter from the food, providing nutrients for microbial growth in the water. Examples of different types of process water are vegetable washers, vegetable cooling baths, poultry chillers, and meat washers.

Given the nature of the food as well as the presence of sediments and soluble materials, the water, flume, and other transport or processing equipment may be subject to the growth of unwanted microorganisms. These microorganisms are generally undesirable to the food, the water, the flume and may cause buildup on all water contact surfaces of slime or biofilm, which requires frequent cleaning to remove. Because the transport water, process water and equipment are in contact with food products, the control of unwanted microorganisms presents certain problems created by a food contact environment containing microorganisms. The disinfectant compositions may be used as sanitizers in these aqueous food transport and process streams, as well as “clean-in-place” sanitizers and biofilm removers for the processing equipment.

The advantageous properties of this invention can be observed by reference to the following examples, which illustrate but do not limit the invention.

### EXAMPLES

### General Procedures

### Preparation of the Peracid/chlorine Dioxide Compositions

Peracetic acid/chlorine dioxide solutions were prepared by mixing a solution of 5% peracetic acid with a solution of 2% stabilized chlorine dioxide (OXINE® stabilized chlorine dioxide, Bio-Cide International, Norman, Okla.; pH=8.5) in various proportions. The 5% peracetic solution was an equilibrium mixture containing hydrogen peroxide (about 22%) and acetic acid. The mixture was allowed to react for a predetermined time and then diluted with distilled water to a solution that contained the desired amount of peracetic acid. Dilution effectively stops formation of chlorine dioxide.

The following ratios were evaluated (peracetic acid solution to stabilized chlorine dioxide solution): 4:1, 3:1, 2:1, 1:1, 1:2, 1:3, and 1:4. Dilution was carried out after either 0.5 min or 3 min. To determine the concentration of chlorine dioxide, a set of sample was prepared and analyzed for chlorine dioxide by spectrophotometry at 360 nm using standard analytical techniques. See, for example, The Chlorine Dioxide Handbook, D. J. Gates, American Water Works Association, Denver, 1998, pp. 116-118. The results are given in Table 1. The concentration of chlorine dioxide in each solution evaluated was calculated from the data in Table 1 and the dilution factor used to each sample to a solution that contained about 50 ppm of peracetic acid.

TABLE 1

| Ratio^{a} | % PAA | [ClO₂] after 0.5 min | [ClO₂] after 3.0 min |
|---|---|---|---|
| 4:1 | 4.0% | 27 | 113 |
| 3:1 | 3.75% | 32 | 140 |
| 2:1 | 3.33% | 37 | 174 |
| 1:1 | 2.50% | 40 | 198 |
| 1:2 | 1.67% | 36 | 154 |
| 1:3 | 1.25% | 28 | 115 |
| 1:4 | 1.0% | 20 | 93 |

^{a}5% PAA to 2% stabilized chlorine dioxide.

The procedure was repeated with 5% peracetic acid to which about 0.5% sulfuric acid had been added. The results are given in Table 2.

TABLE 2

| Ratio^{a} | % PAA | [ClO₂] after 0.5 min | [ClO₂] after 3.0 min |
|---|---|---|---|
| 4:1 | 4.12% | 63 | 144 |
| 3:1 | 3.75% | 80 | 283 |
| 2:1 | 3.33% | 150 | 525 |
| 1:1 | 2.50% | 129 | 432 |
| 1:2 | 1.71% | 90 | 324 |
| 1:3 | 1.29% | 56 | 202 |
| 1:4 | 1.03% | 56 | 186 |

^{a}5% PAA to 2% stabilized chlorine dioxide.

Solutions that comprise about 50 ppm of PAA also comprise about 220 ppm of hydrogen peroxide. Solutions that comprise about 25 ppm of PAA also comprise about 110 ppm of hydrogen peroxide.

### Evaluation Procedure

Peracetic acid/chlorine dioxide solutions were evaluated by a modified version of the IsoGrid Hydrophobic Grid Membrane Filter [HGMF] Disinfectant Test using pathogens that had been surface dried on filters. The procedure is described in “Comparative Biocidal Capacities of Oxidative and Non-Oxidative Sanitizers vs. L. monocytogens, E. coli 0157:H7, and Salmonella typhimurium Using a Modified Surface Dried Film Assay Method,” C. J. Giambrone, G. Diken, and J. Lalli, Abstracts of the Annual Meeting of the International Association for Food Protection, Atlanta, August, 2000, using polycarbonate filters in an IsoGrid HGMF. The contact time, the time the bacteria were exposed to the disinfectant composition, was one minute. After 1 minute, the disinfectant composition was neutralized by addition of 0.5% thiosulfate in letheen broth. After the samples were plated, visible colonies were counted and converted into log₁₀ using conventional techniques. The log₁₀ reduction was determined by subtracting each measured value from the positive control.

### Example 1

Peracetic acid/chlorine dioxide solutions were prepared by mixing a solution of 5% peracetic acid with a solution of 2% stabilized chlorine dioxide (OXINE® stabilized chlorine dioxide, Bio-Cide International, Norman, Okla.; pH=8.5) in the proportions indicated below. The mixtures were allowed to react for either 0.5 min or 3 min and then diluted with water to solutions that contained about 50 ppm of peracetic acid. The solutions were evaluated against Staphylococcus aurens ATCC 6538 using the procedure described above. Evaluation of the solutions that were allowed to react for 0.5 min is given in Table 3. Evaluation of the solutions that were allowed to react for 3 min is given in Table 4.

TABLE 3
Evaluation using Staphylococcus aurens ATCC 6538
0.5 min generation time and 1 min contact time

| | | log₁₀ | log₁₀ |
|---|---|---|---|
| Ratio | Concentrations^{a} | recovery | reduction |
| — | Control | 5.29 | — |
| — | 50 ppm PAA^{b} | 2.53 | 2.76 |
| 4:1 | 46 ppm PAA + 0.03 ppm ClO₂ | 2.77 | 2.52 |
| 3:1 | 48 ppm PAA + 0.04 ppm ClO₂ | 1.41 | 3.88 |
| 2:1 | 48 ppm PAA + 0.05 ppm ClO₂ | 2.53 | 2.76 |
| 1:1 | 50 ppm PAA + 0.09 ppm ClO₂ | 1.62 | 3.66 |
| 1:2 | 48 ppm PAA + 0.11 ppm ClO₂ | 2.25 | 3.04 |
| 1:3 | 48 ppm PAA + 0.10 ppm ClO₂ | 0.66 | 4.63 |
| 1:4 | 48 ppm PAA + 0.14 ppm ClO₂ | TNTC^{c} | — |

^{a}Chlorine dioxide concentrations calculated from the data in Table 1.
^{b}All PAA solutions also contain about 220 ppm of H₂O₂.
^{c}Too numerous to count.

TABLE 4
Evaluation using Staphylococcus aurens ATCC 6538
3 min generation time and 1 min contact time

| | | log₁₀ | log₁₀ |
|---|---|---|---|
| Ratio | Concentrations^{a} | recovery | reduction |
| — | Control | 5.29 | — |
| — | 50 ppm PAA^{b} | 2.53 | 2.76 |
| 4:1 | 46 ppm PAA + 0.14 ppm ClO₂ | 2.56 | 2.73 |
| 3:1 | 48 ppm PAA + 0.19 ppm ClO₂ | 2.43 | 2.86 |
| 2:1 | 48 ppm PAA + 0.26 ppm ClO₂ | 2.33 | 2.96 |
| 1:1 | 50 ppm PAA + 0.40 ppm ClO₂ | 0.92 | 4.37 |
| 1:2 | 48 ppm PAA + 0.46 ppm ClO₂ | 1.85 | 3.44 |
| 1:3 | 48 ppm PAA + 0.46 ppm ClO₂ | 0.90 | 4.39 |
| 1:4 | 48 ppm PAA + 0.47 ppm ClO₂ | 1.70 | 3.59 |

^{a}Chlorine dioxide concentrations calculated from the data in Table 1.
^{b}All PAA solutions also contain about 220 ppm of H₂O₂.

### Example 2

The procedure of Example 1 was repeated with Enterobacter aerogenes ATCC 15038. Evaluation of the solutions that were allowed to react for 0.5 min are given in Table 5. Evaluation of the solutions that were allowed to react for 3 min is given in Table 6.

TABLE 5
Evaluation using Enterobacter aerogenes ATCC 15038
0.5 min generation time and 1 min contact time

| | | log₁₀ | log₁₀ |
|---|---|---|---|
| Ratio | Concentrations^{a} | recovery | reduction |
| — | Control | 6.44 | — |
| — | 50 ppm PAA^{b} | 1.62 | 4.82 |
| 4:1 | 46 ppm PAA + 0.03 ppm ClO₂ | 1.46 | 4.98 |
| 3:1 | 48 ppm PAA + 0.04 ppm ClO₂ | 0.97 | 5.47 |
| 2:1 | 48 ppm PAA + 0.05 ppm ClO₂ | 1.06 | 5.38 |
| 1:1 | 50 ppm PAA + 0.08 ppm ClO₂ | 0.86 | 5.58 |
| 1:2 | 48 ppm PAA + 0.11 ppm ClO₂ | 0.54 | 5.90 |
| 1:3 | 48 ppm PAA + 0.11 ppm ClO₂ | 1.04 | 5.40 |
| 1:4 | 48 ppm PAA + 0.14 ppm ClO₂ | TNTC^{c} | — |

^{a}Chlorine dioxide concentrations calculated from the data in Table 1.
^{b}All PAA solutions also contain about 220 ppm of H₂O₂.
^{c}Too numerous to count.

TABLE 6
Evaluation using Enterobacter aerogenes ATCC 15038
3 min generation time and 1 min contact time

| | | log₁₀ | log₁₀ |
|---|---|---|---|
| Ratio | Concentrations^{a} | recovery | reduction |
| — | Control | 6.44 | — |
| — | 50 ppm PAA^{b} | 1.62 | 4.82 |
| 4:1 | 46 ppm PAA + 0.14 ppm ClO₂ | 0.99 | 5.45 |
| 3:1 | 48 ppm PAA + 0.19 ppm ClO₂ | 0.88 | 5.56 |
| 2:1 | 48 ppm PAA + 0.26 ppm ClO₂ | 1.22 | 5.22 |
| 1:1 | 50 ppm PAA + 0.40 ppm ClO₂ | 0.73 | 5.71 |
| 1:2 | 48 ppm PAA + 0.46 ppm ClO₂ | 0.63 | 5.81 |
| 1:3 | 48 ppm PAA + 0.46 ppm ClO₂ | 0.84 | 5.60 |
| 1:4 | 48 ppm PAA + 0.47 ppm ClO₂ | 0.77 | 5.57 |

^{a}Chlorine dioxide concentrations calculated from the data in Table 1.
^{b}All PAA solutions also contain about 220 ppm of H₂O₂.

### Example 3

The procedure of Example 1 was repeated with 5% peracetic acid to which about 0.5% sulfuric acid had been added. The results for Staphylococcus aurens ATCC 6538 and Enterobacter aerogenes ATCC 15038 are given in Table 7 and Table 8, respectively.

TABLE 7
Evaluation using Staphylococcus aurens ATCC 6538
0.5 min generation time and 1 min contact time

| | | log₁₀ | log₁₀ |
|---|---|---|---|
| Ratio | Concentrations^{a} | recovery | reduction |
| — | Control | 5.02 | — |
| — | 50 ppm PAA^{b} | 0.63 | 4.39 |
| 3:1 | 50 ppm PAA + 0.11 ppm ClO₂ | 1.02 | 4.00 |
| 2:1 | 50 ppm PAA + 0.23 ppm ClO₂ | 0.39 | 4.63 |
| 1:2 | 48 ppm PAA + 0.27 ppm ClO₂ | 0.15 | 4.87 |
| 1:3 | 50 ppm PAA + 0.22 ppm ClO₂ | 0.0 | 5.02 |

^{a}Chlorine dioxide concentrations calculated from the data in Table 2.
^{b}All PAA solutions also contain about 220 ppm of H₂O₂.

TABLE 8
Evaluation using Enterobacter aerogenes ATCC 15038
0.5 min generation time and 1 min contact time

| | | log₁₀ | log₁₀ |
|---|---|---|---|
| Ratio | Concentrations^{a} | recovery | reduction |
| — | Control | 6.80 | — |
| — | 50 ppm PAA^{b} | 0.91 | 5.89 |
| 3:1 | 50 ppm PAA + 0.11 ppm ClO₂ | 0.97 | 5.83 |
| 2:1 | 50 ppm PAA + 0.23 ppm ClO₂ | 0.45 | 6.35 |
| 1:2 | 48 ppm PAA + 0.27 ppm ClO₂ | 0.35 | 6.45 |
| 1:3 | 50 ppm PAA + 0.22 ppm ClO₂ | 0.35 | 6.45 |

^{a}Chlorine dioxide concentrations calculated from the data in Table 2.
^{b}All PAA solutions also contain about 220 ppm of H₂O₂.

### Example 4

The procedure of Example 3 was repeated except that the peracetic acid/chlorine dioxide solutions were prepared by mixing a 5% peracetic acid with about 0.5% sulfuric acid with a solution of 2% stabilized chlorine dioxide was diluted a solution that contained about 25 ppm of peracetic acid. The results are given in Table 9.

TABLE 9
Evaluation using Staphylococcus aurens ATCC 6538
0.5 min generation time and 1 min contact time

| | | log₁₀ | log₁₀ |
|---|---|---|---|
| Ratio | Concentrations^{a} | recovery | reduction |
| — | Control | 5.44 | — |
| — | 25 ppm PAA^{b} | >4.07 | <1.4 |
| 4:1 | 25 ppm PAA + 0.04 ppm ClO₂ | >4.07 | <1.4 |
| 3:1 | 25 ppm PAA + 0.05 ppm ClO₂ | 3.99 | 1.45 |
| 2:1 | 25 ppm PAA + 0.11 ppm ClO₂ | 3.40 | 2.04 |
| 1:1 | 25 ppm PAA + 0.12 ppm ClO₂ | 3.84 | 1.60 |
| 1:2 | 25 ppm PAA + 0.13 ppm ClO₂ | 2.80 | 2.64 |
| 1:3 | 25 ppm PAA + 0.11 ppm ClO₂ | 2.58 | 2.87 |
| 1:4 | 25 ppm PAA + 0.13 ppm ClO₂ | 2.78 | 2.66 |

^{a}Chlorine dioxide concentrations calculated from the data in Table 2.
^{b}All PAA solutions also contain about 110 ppm of H₂O₂.

### Example 5

The procedure of Example 4 was repeated except that the peracetic acid solution and the peracetic acid/chlorine dioxide solutions were diluted with tap water instead of distilled water. The results are given in Table 10.

TABLE 10
Evaluation using Staphylococcus aurens ATCC 6538
0.5 min generation time and 1 min contact time
tap water used to generate the disinfectant composition

| | | log₁₀ | log₁₀ |
|---|---|---|---|
| Ratio | Concentrations^{a} | recovery | reduction |
| — | Control | 5.44 | — |
| — | 25 ppm PAA^{b} | 3.96 | 1.48 |
| 2:1 | 25 ppm PAA + 0.11 ppm ClO₂ | 4.07 | 1.37 |
| 1:1 | 25 ppm PAA + 0.12 ppm ClO₂ | 3.86 | 1.58 |
| 1:2 | 25 ppm PAA + 0.13 ppm ClO₂ | 3.84 | 1.60 |

^{a}Chlorine dioxide concentrations calculated from the data in Table 2.
^{b}All PAA solutions also contain about 110 ppm of H₂O₂.

### Example 6

The procedure of Example 3 was repeated using a solution of 2% sodium chlorite and a solution of 2% stabilized chlorine dioxide. The solutions were allowed to react for 0.5 min before dilution. The results are given in Tables 11 and 12.

TABLE 11
Evaluation using Staphylococcus aurens ATCC 6538
50 ppm PAA

| Ratio | | log₁₀ recovery | log₁₀ reduction |
|---|---|---|---|
| — | Control | 5.59 | — |
| 1:2 | Stabilized ClO₂ | 0.86 | 4.73 |
| 1:2 | Sodium chlorite | 1.23 | 4.36 |

TABLE 12
Evaluation using Listeria monocytogenes
50 ppm PAA

| Ratio | | log₁₀ recovery | log₁₀ reduction |
|---|---|---|---|
| — | Control | 6.78 | — |
| 1:1 | Stabilized ClO₂ | 1.32 | 5.46 |
| 1:1 | Sodium chlorite | 2.34 | 4.45 |
| 1:2 | Stabilized ClO₂ | 2.23 | 4.55 |
| 1:2 | Sodium chlorite | 2.28 | 4.51 |
| 1:3 | Stabilized ClO₂ | 1.89 | 4.89 |
| 1:3 | Sodium chlorite | 2.02 | 4.76 |

### Comparative Examples

Using the procedures described above, peracetic acid and chlorine dioxide were individually evaluated with Listeria monocytogenes, which is analogous to Staphylococcus aurens, and Salmonella typhimurium, which is analogous to Enterobacter aerogenes. Chlorine dioxide was generated by mixing 1 part of 75% phosphoric acid to 9 parts of OXINE® stabilized chlorine dioxide and diluting the resulting mixture to produce a 5 ppm chlorine dioxide solution. PAA was prepared by diluting 5% PAA to produce a 50 pmm PAA solution. The concentration of chlorine dioxide was measure spectrophotometrically at 360 nm. The results are given in Tables 13 and 14.

TABLE 13
Comparative evaluation using Listeria monocytogenes

| Biocide | Control titer | log₁₀ recovery | log₁₀ reduction |
|---|---|---|---|
| 5 ppm ClO₂ | 6.5 | 3.3 | −3.2 |
| 50 ppm PAA^{a} | 6.5 | 2.2 | −4.3 |

^{a}From 5% PAA - contains about 220 ppm of H₂O₂.

TABLE 14
Comparative evaluation using Salmonella typhimurium

| Biocide | Control titer | log₁₀ recovery | log₁₀ reduction |
|---|---|---|---|
| 5 ppm ClO₂ | 6.75 | 2.75 | −4.0 |
| 50 ppm PAA^{a} | 6.75 | 2.28 | −4.5 |

^{a}From 5% PAA - contains about 220 ppm of H₂O₂.

Having described the invention, we now claim the following and their equivalents.

What is claimed is:

1. A disinfectant composition comprising:
(a) water;
(b) about 10 ppm to about 500 ppm of a lower organic peracid; and
(c) about 0.1 ppm to about 20 ppm of chloride dioxide.

2. The composition of claim 1 in which the peracid is peracetic acid.

3. The composition of claim 1 in which the composition additionally comprises about 5 ppm to about 5000 ppm of hydrogen peroxide.

4. The composition of claim 3 in which the composition comprises about 10 ppm to about 200 ppm of the lower organic peracid and about 0.4 ppm to about 2 ppm of chlorine dioxide.

5. The composition of claim 4 in which the peracid is peracetic acid.

6. The composition of claim 4 in which the peracid is a mixture of peracetic acid and one or more peracids selected from the group consisting of aliphatic monocarboxylic peracids having 3 to 10 carbon atoms.

7. The composition of claim 4 in which the peracid is a mixture of peracetic acid and peroctanoic acid.

8. The composition of claim 7 in which the hydrogen peroxide concentration is about 10 ppm to about 1000 ppm.

9. The composition of claim 7 in which the hydrogen peroxide concentration is about 50 ppm to about 500 ppm.

10. A method for disinfecting an object, the method comprising:
applying a disinfectant composition to the object, the disinfectant composition comprising:
(a) water;
(b) about 10 ppm to about 500 ppm of a lower organic peracid; and
(c) about 0.1 ppm to about 20 ppm of chlorine dioxide.

11. The method of claim 10 in which the object is selected from the group consisting of animal carcasses, meat products, fruits, and vegetables.

12. The method of claim 10 in which the peracid is peracetic acid.

13. The method of claim 10 in which the composition additionally comprises about 5 ppm to about 5000 ppm of hydrogen peroxide.

14. The method of claim 13 in which the composition comprises about 10 ppm to about 200 ppm of the lower organic peracid and about 0.4 ppm to about 2 ppm of chlorine dioxide.

15. The method of claim 14 in which the peracid is peracetic acid.

16. The method of claim 14 in which the peracid is a mixture of peracetic acid and one or more peracids selected from the group consisting of aliphatic monocarboxylic peracids having 3 to 10 carbon atoms.

17. The method of claim 14 in which the peracid is a mixture of peracetic acid and peroctanoic acid.

18. The method of claim 14 in which the object is selected from the group consisting of animal carcasses, meat products, fruits, and vegetables.

19. The method of claim 14 in which the object is a food contact surface.

20. A method for preparing a disinfectant composition, the method comprising:
(a) mixing an aqueous solution of a lower organic peracid and a chlorite to form a mixture;
(b) allowing the mixture to stand for a predetermined period of time; and
(c) diluting the mixture with water to form the disinfectant composition, the disinfectant composition comprising:
(i) water;
(ii) about 10 ppm to about 500 ppm of a lower organic peracid;
(iii) about 0.1 ppm to about 20 ppm of chlorine dioxide; and
(d) about 5 ppm to about 5000 ppm of hydrogen peroxide.

21. The method of claim 20 in which the lower organic peracid is peracetic acid.

22. The method of claim 20 in which the chlorite is sodium chlorite.

23. The method of claim 20 in which the aqueous solution of the lower organic peracid comprises about 5% to about 35% by weight of the lower organic peracid.

24. The method of claim 23 in which the lower organic peracid is peracetic acid.

25. The method of claim 22 in which the predetermined period of time is about 0.25 min to about 5 min.

26. The method of claim 22 in which the composition comprises about 10 ppm to about 200 ppm of the lower organic peracid and about 0.4 ppm to about 2 ppm of chlorine dioxide.

27. The method of claim 26 in which the peracid is peracetic acid.

28. The method of claim 26 in which the peracid is a mixture of peracetic acid and one or more peracids selected from the group consisting of aliphatic monocarboxylic peracids having 3 to 10 carbon atoms.

29. The method of claim 26 in which the predetermined period of time is about 0.25 min to about 5 min.

30. A method for treating water in a water system, the method comprising:
(a) mixing an aqueous solution of a lower organic peracid and a chlorite to form a mixture;
(b) allowing the mixture to stand for a predetermined period of time; and
(c) adding the mixture to the water in the water system so that the water in the water system comprises:
(i) about 10 ppm to about 500 ppm of a lower organic peracid;
(ii) about 0.1 ppm to about 20 ppm of chlorine dioxide; and
(iii) about 5 ppm to about 5000 ppm of hydrogen peroxide.

31. The method of claim 30 in which the lower organic peracid is peracetic acid.

32. The method of claim 30 in which the chlorite is sodium chlorite.

33. The method of claim 32 in which the aqueous solution of the lower organic peracid comprises about 5% to about 35% by weight of the lower organic peracid.

34. The method of claim 33 in which the lower organic peracid is peracetic acid.

35. The method of claim 32 in which the predetermined period of time is about 0.25 min to about 5 min.

36. The method of claim 32 in which the composition comprises about 10 ppm to about 200 ppm of the lower organic peracid and about 0.4 ppm to about 2 ppm of chlorine dioxide.

37. The method of claim 36 in which the peracid is peracetic acid.

38. The method of claim 36 in which the peracid is a mixture of peracetic acid and one or more peracids selected from the group consisting of aliphatic monocarboxylic peracids having 3 to 10 carbon atoms.

39. The method of claim 36 in which the predetermined period of time is about 0.25 min to about 5 min.

40. The method of claim 37 in which the aqueous solution of the lower organic peracid comprises about 4% to about 8% of peracetic acid.

41. The method of claim 40 in which the hydrogen peroxide concentration is about 50 ppm to about 500 pmm.
